(19) United States
(12) Patent Application Publication
(10) Pub. No.: US 2017/0189649 A1
(43) Pub. Date: Jul. 6, 2017

(54) POST CERVICAL ARTIFICIAL INSEMINATION CATHETER

(71) Applicant: COMMON SENSE INSEMINATION PRODUCTS CO., LLC., Henderson, NV (US)

(72) Inventors: MARK E. ANDERSON, HENDERSON, NV (US); JO ANN CATEDRAL HACHUELA, TACURONG CITY (PH)

(73) Assignee: COMMON SENSE INSEMINATION PRODUCTS CO., LLC., HENDERSON, NV (US)

(21) Appl. No.: 15/397,745

(22) Filed: Jan. 4, 2017

Related U.S. Application Data
(60) Provisional application No. 62/281,736, filed on Jan. 22, 2016.
(60) Provisional application No. 62/275,231, filed on Jan. 5, 2016.

(51) Int. Cl. A61M 25/06

(52) U.S. Cl. CPC A61M 25/0643; A61M 25/06; A61M 2210/1425; A61M 2202/04; A61M 2202/0007

(57) ABSTRACT

A post-cervical insemination catheter assembly including a cannula; a guide catheter slideably positioned about said cannula; and, a tip element securely connected to a distal end of said cannula. The tip element is configured to receive fluid from the cannula and dispense the fluid from a tip element outlet end. The tip element outlet end remains closed when loading the cannula through a vaginal canal and when traversing a cervix of the animal and then automatically opens when sufficient fluid pressure in the tip element is applied, thus enhancing injection of fluid to the utero-tubal junction, bio-secure delivery of fluids from any attached container, and a much safer less invasive experience for the recipient then prior post-cervical artificial insemination (PCAI) catheter assemblies.

### CROSS REFERENCE TO RELATED APPLICATIONS

This application claims the benefit of U.S. Provisional Application Ser. Nos. 62/275,231 filed on Jan. 5, 2016 and 62/281,736 filed on Jan. 22, 2016, the entire contents of which are hereby incorporated herein by reference thereto.

### BACKGROUND OF THE INVENTION

1. Field of the Invention

The present invention relates generally to post-cervical artificial insemination (PCAI) or post-cervical embryo transfer (PCET) of animals and more particularly to a post-cervical catheter assembly providing enhanced injection of fluid to the utero-tubal junction.

2. Description of the Related Art

Most post-cervical artificial insemination catheters (PCAI) use tips that fit inside a very small piece of tubing, i.e. a cannula. These tips are usually hard and they have openings that collect contamination when inserted through the vaginal canal, the guide catheter, and/or the cervix of the animal. This is problematic because one of the main functions of the cervix is to trap and sanitize any foreign matter from entering the reproductive tract. PCAI bypasses the cervix, so it is desirable to maintain bio-security from the semen container into the uterus. Present inventor, Mark Anderson, is a co-inventor of multiple patents entitled “METHOD AND APPARATUS FOR CREATING A PATHWAY IN AN ANIMAL” (U.S. Pat. Nos. 6,526,916, 6,662,750, 8,860,235, 7,343,875, 7,647,891, 7,971,553, 8,136,483). Absolute Swine Insemination Co, LLC provides a solution to this bio-security problem with their AMG™ Series catheters using an inverting latex membrane.

Another problem with traditional PCAI catheters is they are inherently invasive. Hard tips with relatively flexible cannulas can cause damage to delicate cervical tissues and in some cases, puncture the cervix, uterine walls, cysts, etc. No matter how flexible the cannula is, they do not solve this problem. Small diameter rigid tip based cannulas, can easily get caught and/or trapped in the cervical folds. AI technicians that push too hard on the cannulas can seriously hurt the animals.

Existing cannulas have small inside diameters preventing the required flow of genetic material into the reproductive tract like the AMG™ Series. Adding to the cannulas restrictive flow, the small hard tips that are inserted inside the cannula reduce the flow to approximately 37% of a traditional inner cannula's initial volume capabilities due to the barbed end's inner diameter of approximately 47/1000″ (1.20 mm) being inserted into the cannula. To make matters worse, usually the inserted tips redirect the flow of semen sideways out of two or four extremely small cavities. This prevents the AI technician's ability to inject semen into the uterine horns and up to the utero-tubal junction with any speed or ease. No matter how hard one squeezes a semen container, the restrictive cannula, combined with the further reduction of the hard tip, coupled with diverting and or changing direction of the semen in the tip to exit “sideways”, results in a dribble of semen into the uterus with limited injection capabilities.

Additional flow restrictions are created with openings in the spouts of semen containers. Small exit holes, many times less than the ID of the cannula are used that create turbulence when semen is squeezed under pressure. Said turbulence can break semen tails and render them ineffective for fertilization.

Currently, rubberized ends (called handles) are needed on the guide catheters and/or cannulas to accept the spouts of semen containers.

Most PCAI cannula tips do nothing more than provide a rounded surface as compared to just using the open end of a cut cannula; they assist in providing a blunt, relatively smooth surface coming in contact with delicate tissues but remain invasive and intrusive. As used herein the term “PCAI catheter” may, at times, be equally referring to both post-cervical insemination and post-cervical embryo transfer (PCET) catheters. Many cannula tips are used to simply cover up the sharp, cut edge of the cannula. They are far smaller in diameter compared to a boar's penis, and they have been designed out of convenience to manufacture and fit through traditional catheter tubes. Current PCAI tips do not assist in guiding the catheter safely through the cervix; their current designs are actually counterproductive to that.

Another problem with traditional PCAI catheters is that most cannulas are not held firmly in place and can fall out of the guide catheter during use. Several “soft tipped” cannulas have entered the market, but their expected performance has been counter-productive to the intended results. An example of such a “soft tipped” device is disclosed in Chinese Pat. No. 205626184U (ZL201520835886.1). Soft tipped versions tend to fold over and create a bent over mass at the end of the cannula hampering forward progression, and their softness properties cause them to act like a pencil eraser on paper. Instead of providing additional protection and safety, they can grab the cervical folds and cause greater friction.

### SUMMARY OF THE INVENTION

In one aspect, the present invention is post-cervical insemination catheter assembly including a cannula; a guide catheter slideably positioned about said cannula; and, a tip element securely connected to a distal end of said cannula. The tip element is configured to receive fluid from the cannula and dispense the fluid from a tip element outlet end. The tip element outlet end remains closed when loading the cannula through the vaginal canal and when traversing the cervix and then automatically opens when sufficient fluid pressure in the tip element is applied, thus enhancing injection of fluid to the utero-tubal junction. This provides a continual flow of bio-secure, uncontaminated semen directly from the semen container into the reproductive tract.

In one aspect, the catheter assembly is embodied as a post-cervical artificial insemination (PCAI) catheter assembly. In another aspect the catheter assembly is embodied as a post-cervical embryo transfer (PCET) catheter assembly.

In another aspect, the present invention is embodied as a method for providing artificial insemination of an animal including the steps of: a) providing a post-cervical insemination catheter assembly having: i) a cannula; ii) a guide catheter slideably positioned about said cannula; and, iii) a tip element securely connected to a distal end of said cannula configured to receive fluid from the cannula and dispense the fluid from a tip element outlet end; b) inserting the guide catheter through a vaginal canal of an animal and locking into a cervix of the animal; c) pushing the cannula through the guide catheter to a desired position in the reproductive tract; and, d) applying sufficient fluid pressure in the tip element to automatically open the tip element outlet end enhancing injection of fluid to the utero-tubal junction. Bio-security of fluids being transferred through the catheter assembly is maintained.

In another broad aspect, the invention is embodied as a post-cervical insemination catheter assembly, including: a foam element; a hard insert; a tip element; and a catheter tube. The foam element has an inner surface. The hard insert has an outer surface secured to the inner surface of the foam element. The hard insert includes an axially extending elongated body and at least one locking rib extending from an inner surface of the axially extending elongated body. The tip element includes a distal nub portion and a proximal nub portion including at least a first proximal nub and at least a first associated proximal nub recess. The catheter tube has a distal end securely connected to the tip element. When the tip element is locked into a position in the foam element the catheter tube can be pushed and/or pulled through a vaginal canal of an animal providing locking into a cervix of the animal. When the catheter tube is rotated the catheter tube can move relative to the foam element allowing it to traverse the cervix of the animal. A tip element outlet end remains closed when loading the catheter tube through the vaginal canal and when traversing the cervix. The tip element outlet and automatically opens when sufficient fluid pressure in the tip element is applied, thus enhancing injection of fluid to a utero-tubal junction of the animal. In one embodiment the catheter assembly is a post-cervical artificial insemination (PCAI) catheter assembly. In another embodiment the catheter assembly is a post-cervical embryo transfer (PCET) catheter assembly.

In another broad aspect, the invention is embodied as a post-cervical insemination catheter assembly, including a cannula; a handle and a tip element. The handle is connected to the cannula and is configured to provide attachment of a container to the cannula. The tip element includes a proximal portion securely attached to the cannula; a main ribbed portion extending from said proximal portion; a bulb shaped distal end; and a recessed portion between the main ribbed portion and a bulb shaped distal end. The recessed portion provides a slicing area. The cannula is configured to provide a fluid from a container and the fluid is transferred through said proximal portion, through said main ribbed portion and dispensed through an opening created in a reduced diameter of the inner diameter of the tip element in the vicinity of the recessed portion when the bulb shaped distal end opens. The bulb shaped distal end remains seated to the main ribbed portion when loading the cannula through a vaginal canal and when traversing a cervix of an animal and then automatically opens when sufficient fluid pressure in the tip element is applied. Thus, injection of the fluid to a utero-tubal junction of the animal is enhanced and bio security of fluids being transferred through the catheter assembly is maintained.

In certain embodiments, the present invention includes a tip member with suitable flexural strength characteristics providing the ability to adequately bend and safely deflect off delicate tissues while maintaining enough structural integrity to hold its original shape. In short, the tip should keep facing forward and never reverse position by folding over upon itself creating an obstruction as compared to a solution. In these embodiments, the tip is semi-flexible and smooth, but also non-absorbent and slippery when coated with cervical and vaginal fluids. There are rounded (i.e. radius) edges that are designed to deflect off the cervical bumps and other obstructions helping to create a safe pathway into the uterus of the animal. The proper flexibility and shape of the tip member allows the catheter to be safely guided through the cervix. It does not stick to the folds and get trapped the way “soft” tips made from silicone and other rubbers can.

Other objects, advantages, and novel features will become apparent from the following detailed description of the invention when considered in conjunction with the accompanying drawings.

### BRIEF DESCRIPTION OF THE DRAWINGS

FIG. 1 is a perspective view of a first embodiment of the catheter assembly, in which tip element includes ribs and a breakaway membrane, the tip element outlet end being shown in a closed position.

FIG. 2 is an enlarged view of a portion of the first embodiment, showing the distal end of the cannula and the tip element, the tip element being shown open.

FIG. 3 is another embodiment in which the outside diameter of the tip element has portions greater than the outside diameter of the guide catheter.

FIG. 4 is another embodiment of tip element which includes a slit on the tip element outlet end.

FIG. 5 is another embodiment of the tip element with a dual taper.

FIG. 6 is another embodiment of the tip element having a continual taper down.

FIG. 7 is an enlarged view of the distal end of another embodiment of the catheter assembly utilizing a catheter tube and a hard insert.

FIG. 8A illustrates the tip element of the embodiment of FIG. 7.

FIG. 8B is a view taken along line 8B-8B of FIG. 8A.

FIG. 8C is a view of the FIG. 7 embodiment with the foam element removed and in an unlocked position.

FIG. 9A shows the hard insert.

FIG. 9B is a schematic cross-section of the tip element within the hard insert, in an unlocked position, as in FIG. 9A.

FIG. 10A illustrates the tip element shown in FIG. 8A rotated 180 degrees.

FIG. 10B is a schematic cross-section of the tip element within the hard insert, in a locked position, as in FIG. 10A.

FIG. 11A is a perspective view, partially in cross-section of the catheter assembly of FIG. 7 embodiment, in a locked position.

FIG. 11B shows the catheter assembly in an initial unlocked position.

FIG. 11C shows the catheter tube and tip element completely forward of the foam element with a stop preventing further forward movement.

FIG. 12A is a perspective view of another tip element with a recessed portion.

FIG. 12B shows the FIG. 12A moving to an open position.

### DETAILED DESCRIPTION OF THE INVENTION

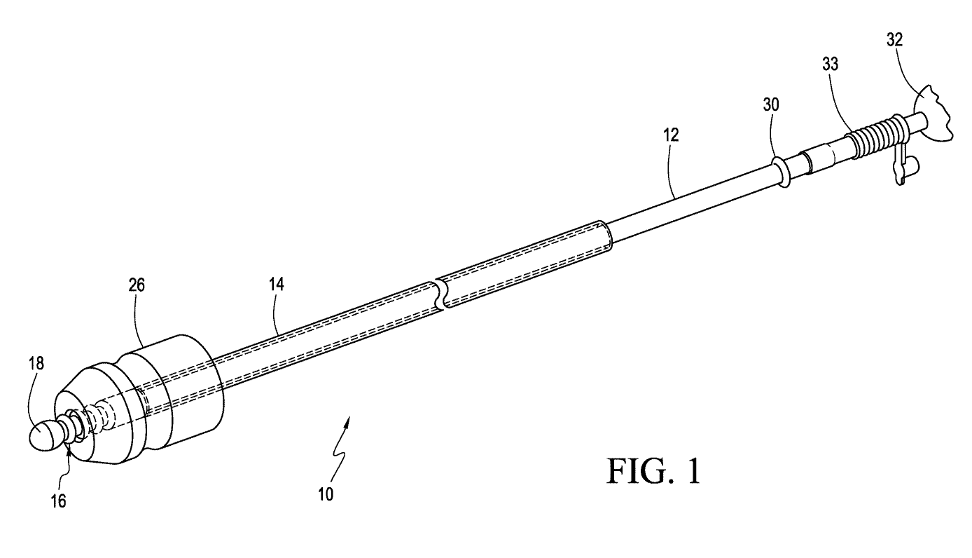

Referring now to the drawings and the characters of reference marked thereon, FIG. 1 illustrates a first embodiment of a PCAI catheter assembly, in accordance with the principles of the present invention, designated generally as 10. The PCAI catheter assembly 10 includes a cannula 12, a guide catheter 14 slideably positioned about the cannula 12, and a tip element 16 securely connected to a distal end of the cannula 12.

The tip element 16 is configured to receive fluid from the cannula and dispense the fluid from a tip element outlet end 18. The tip element outlet end 18 remains closed when loading the cannula through the vaginal canal and when traversing the cervix and then automatically opens when sufficient fluid pressure in the tip element is applied. Thus, enhanced injection of fluid to the utero-tubal junction is provided, and bio-security of fluid passing through the cannula 12 and tip 16 elements is maintained.

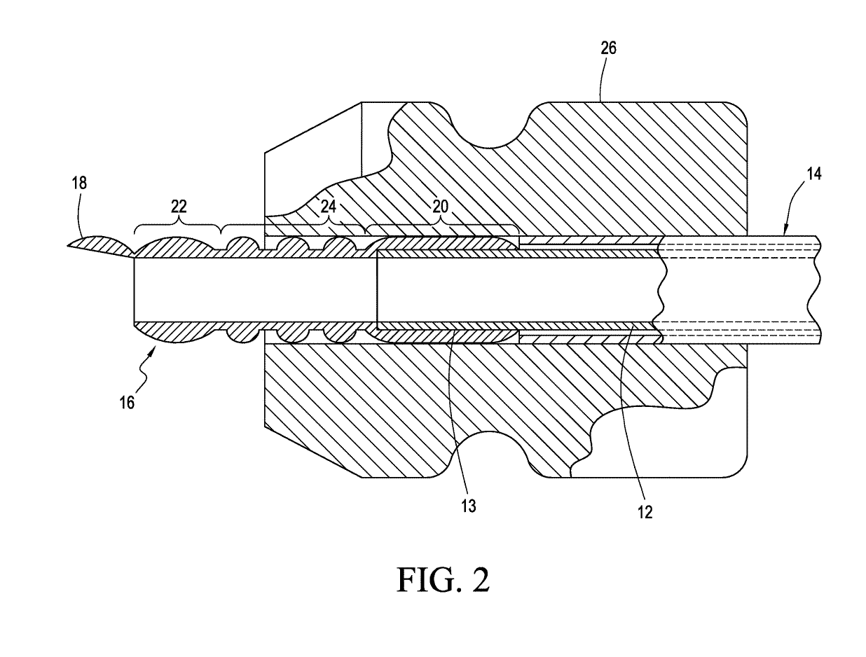

In the embodiment illustrated in FIGS. 1 and 2, the tip element 16 includes a proximal section 20, a distal section 22, and an intermediate section 24 between said proximal section 20 and the distal section 22. The proximal section 20 is substantially cylindrical. The intermediate section 24 is ribbed, and the distal section 22 is bulb-shaped. In this embodiment, the intermediate section 24 and the distal section 22 are substantially equal to the outer diameter of the guide catheter 14. The use of spaced ribs, the proper flexural strength resistance, the proper shore hardness (sometimes referred to as durometer), the proper material selection, and tapering wall thicknesses are all features that cooperate to keep the tip element 16 from collapsing upon itself. The ribs help the tip follow the natural pathway of the animal and guide the cannula behind it.

In this embodiment, the tip element 16 is connected about an outer surface 13 of the cannula 12. Thus, the flow rate is maximized by utilizing the entire 3.2 mm ID of the cannula compared to the 1.2 mm ID of many barbed inserts. The large inside diameter of the tip element 16 combined with a non-restrictive opening at the end of the distal section 22 greatly increases flow compared to barbed tips of the prior art having one or two exit holes per side. The volume percent increase is directly relative to the ID of cannula 12 (and catheter tube 62 in some embodiments).

The foam guide catheter tip 26 provides stability for the catheter assembly 10 relative to the cervix. The tip element 16 rests inside the foam tip of the guide catheter during insertion into the animal.

During operation, the catheter assembly 10 is loaded into the cervix. When the animal relaxes, the cannula is pushed through. It is pushed through until it stops. At this point the tip element 16 (i.e. bulb shaped portion 96 in some embodiments) is in the uterus.

The cannula (or catheter tube 62 in some embodiments) includes a stop mechanism 30 (or 122 in some embodiments) to provide the stop. The stop mechanism may be, for example, a crimped area in the cannula 12 or catheter tube 62 providing a protective stop so the catheter does not enter the reproductive tract too far. An alternative stop mechanism is a heat staked stop. Other alternative stop mechanisms could include the use of separate injection molded pieces that are slid onto the cannula 12, or the common handle used to connect fluid containers.

A fluid container 32 such as a bottle, tube, flat-pack, etc. is attached to a proximal end of the cannula 12 using a handle 33; or, it can attach directly to an outer surface of a proximal end of the cannula 12, eliminating fluid flow restrictions of the fluid container/cannula interfaces of prior catheter assemblies.

When fluid is released from the fluid container 32 at a sufficient pressure, the tip element outlet end 18, in this case configured with a breakaway membrane is opened as shown in FIG. 2. This provides an option to mold the distal end of the tip with an extremely thin area. The membrane is configured to “break” or tear under at the sufficient pressure allowing the flap to open. However, sufficient material remains connected so that it does not stay in the cervix. The increased outside diameter of the tip element and its flexibility are of sufficient size to help open the cervix by gently pushing the folds out of the way and create a pathway when the cannula is pushed through. Backflow is minimized.

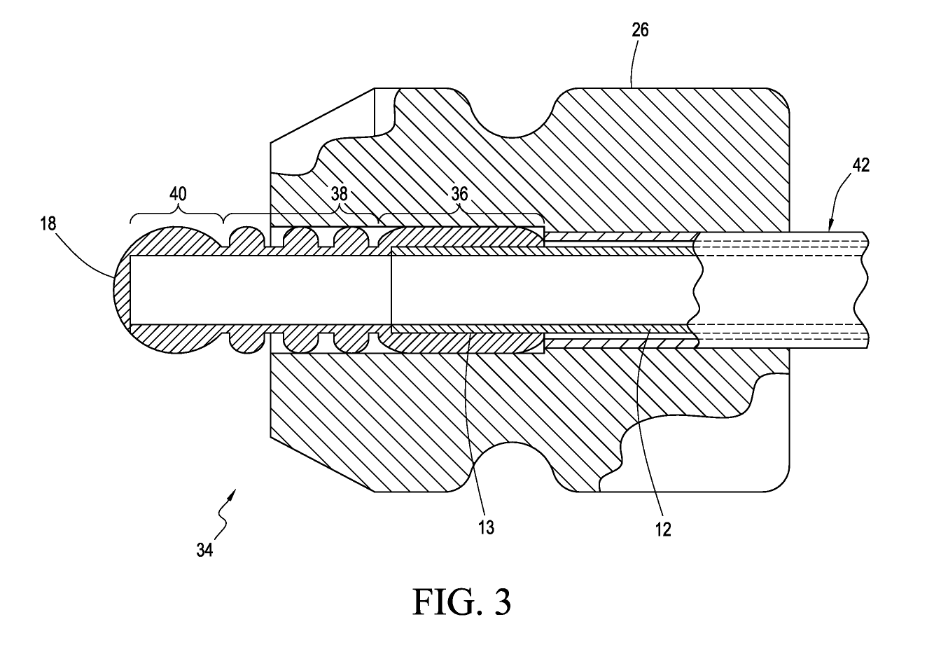

Referring now to FIG. 3, an embodiment 34 is illustrated in which the maximum diameter of the proximal section 36, the intermediate section 38 and the distal section 40 are greater than the outer diameter of the guide catheter 42. Maximizing the diameter of the tip element better emulates the size of a penis of the animal. Also, the resulting assembly has an advantage that the cannula cannot fall out of the guide catheter.

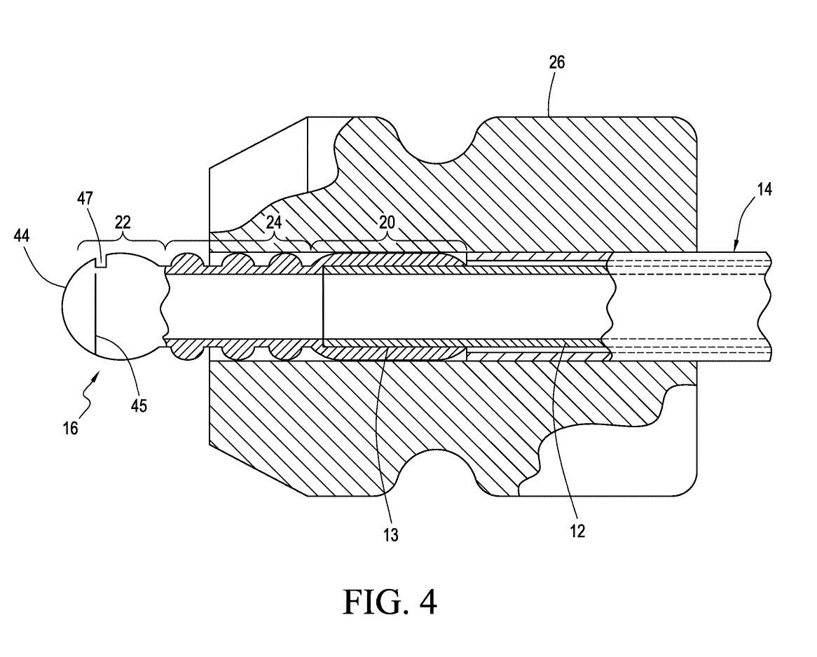

Referring now to FIG. 4, an embodiment is illustrated in which the tip element outlet end 44 includes a slit 45 which opens at a sufficient pressure to allow discharge. Additionally, the tip outlet end is preferably enhanced by way of a bend relief providing unrestricted movement of the flap when sufficient pressure is applied. This bend relief is provided by a recessed portion 47 in the tip element outlet end 44 which provides clearance. The slit is on the side surface of the bulb rather than on the end of the tip outlet end because this provides self-sealing, i.e. less contamination, than if the slit was located at the end. The slits might be slices or molded slits.

Referring now to FIG. 5, a dual taper embodiment 46 is illustrated in which the tip element includes a proximal section 48, a distal section 52, and an intermediate section 50 between the proximal section and the distal section. The proximal section 48 is substantially cylindrical, the intermediate section 50 has a dual taper, and the distal section 52 is bulb-shaped. This embodiment provides flexibility in the midportion with an emphasis on not folding over or collapsing upon itself.

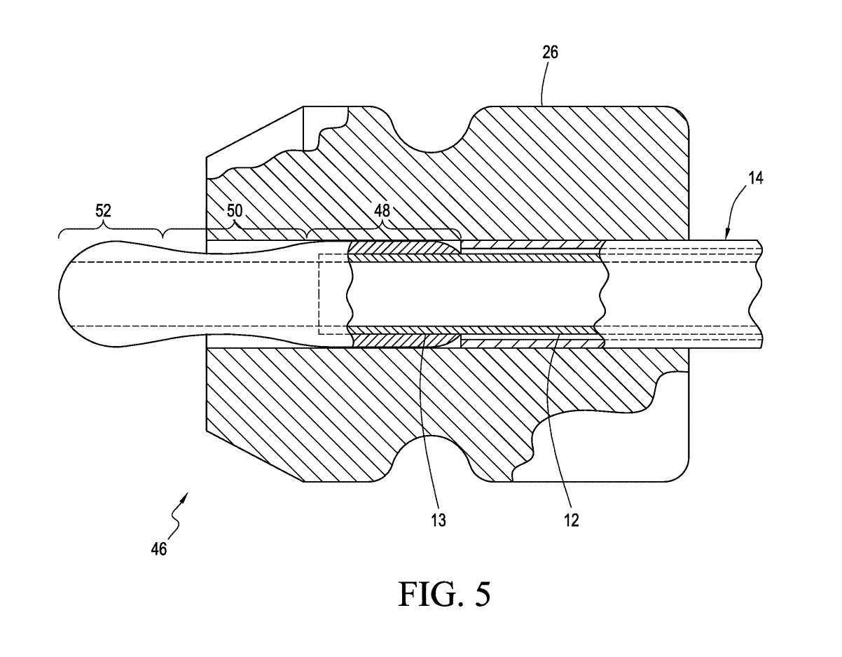

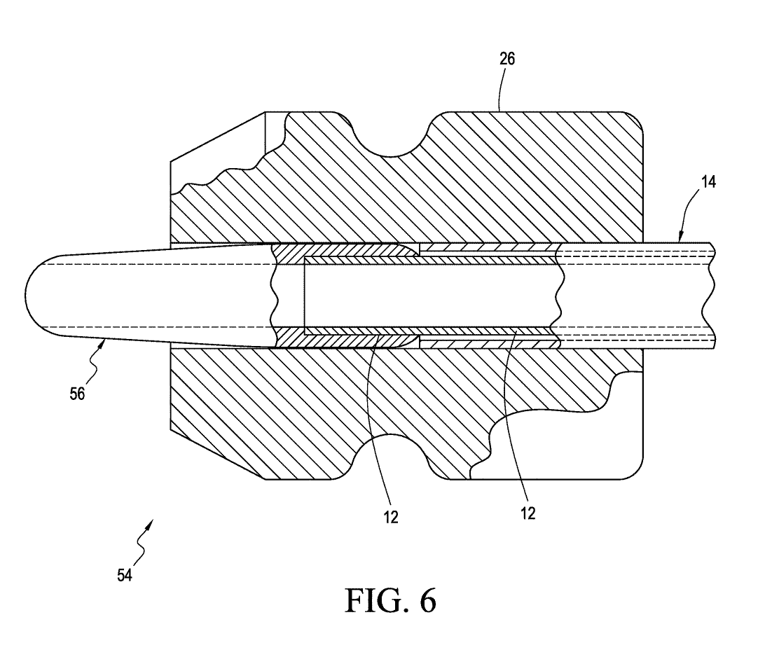

Referring now to FIG. 6, a continual taper embodiment 54 is illustrated in which the tip element 56 has a continual taper down from a proximal section through a distal section. (Therefore, the end of the tip element is more flexible than the more proximal sections thereof.)

The tip element is formed of a semi-flexible material, i.e. with suitable flexural strength characteristics, preferably polyvinyl chloride (PVC). This semi-flexible material allows for adequate elasticity of the tip element to deflect off cervical folds without collapsing upon itself creating additional obstructions and problems. Use of a suitable flexural strength material makes insertion easier than previous catheter assemblies since the semi-flexible tip will guide the catheter into and through the cervix. However, this semi-flexible material has an appropriate shore hardness, and flexural strength characteristics that prevents the tip from folding over and creating a bent over mass at the end of the cannula hampering forward progression. Previous hard tips of the catheters were generally formed of ABS, polypropylene (PP), or polyvinyl chloride (PVC) plastic with high durometer readings. Soft tipped cannulas are generally made of silicone or other extremely soft polymers or rubbers. The semi-flexible tip element of the present invention may be formed of PVC, as mentioned above. As used herein the term “semi-flexible” refers to materials with controllable flexural strength characteristics, an A-shore hardness between 10-90 on the IRHD scale, and preferably within a range of 50 and 70. In a preferred embodiment semi-flexible refers to a shore hardness of 50 providing an optimal flexural strength. Alternatively, instead of being formed of PVC it may be formed of polypropylene (PP), polyethylene (PE), polymers, combinations of polymers, polymers with a range of additives, monomers, rubber, silicone, and/or plastics.

The cannula is preferably formed of polyvinylchloride (PVC).

The tip element includes a tapered wall thickness allowing the tip to bend and follow the natural pathway of the animal, thereby guiding the cannula behind it.

In a preferred embodiment, the tip element is slightly larger than the guide catheter's OD. The tip element OD is preferably no less than the guide catheter ID to lock the cannula in place and keep the cannula 12 from falling out of the guide catheter.

The cannula preferably has an outside diameter just small enough to provide clearance in a guide catheter. In one embodiment, the cannula has an outside diameter of about 11/64 inches (4.37 mm). In this embodiment, the cannula has an inside diameter of about 1/8″ inches (3.2 mm). However, if the guide catheter's diameter is greater, the cannula diameter would/could increase accordingly. In this embodiment, the tip element may have, for example, an outside diameter of 7 mm. The length of the tip element may be about 29 mm. The entire catheter assembly from the proximal end of the cannula to the end of the tip element may be about 22 inches (i.e. about 560 mm).

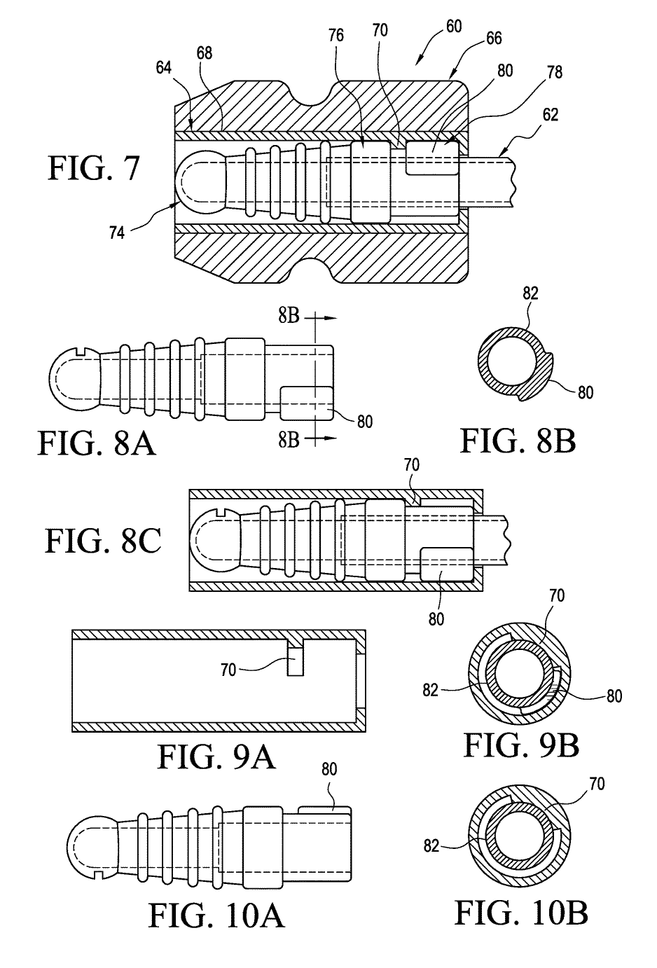

Referring now to FIG. 7 an enlarged view of the distal end of another embodiment of the catheter assembly is illustrated, designated generally as 60 in which a catheter tube 62 and a hard insert 64 are utilized. This embodiment combines the cannula and the guide catheter into one piece, i.e. the catheter tube 62. This distal nub portion 76 goes all the way around the tip element so it cannot pull through the hard insert 64. This assures the foam comes out with the catheter. The hard insert 64 is preferably formed of plastic.

The catheter assembly 60 includes a foam element 66 with an inner surface. The hard insert 64 has an outer surface secured to the inner surface of said foam element 66. The hard insert 64 may be secured to the foam element 66 by, for example, glue or high-frequency welding. The hard insert 64 includes an axially extending elongated body 68 and at least one locking rib (in the instance shown, locking rib 70) extending from an inner surface of the axially extending elongated body 68.

As can be seen by further reference to FIGS. 8A-8C, 9A-9B, and 10A-B, the catheter assembly 60 includes a tip element 74 with a distal nub portion 76 and a proximal nub portion 78. The proximal nub portion 78 includes at least a first proximal nub 80; and at least a first associated proximal nub recess 82. (In, for example, another embodiment (not shown) the proximal nub portion 78 may include a second proximal nub and a second associated proximal nub recess.)

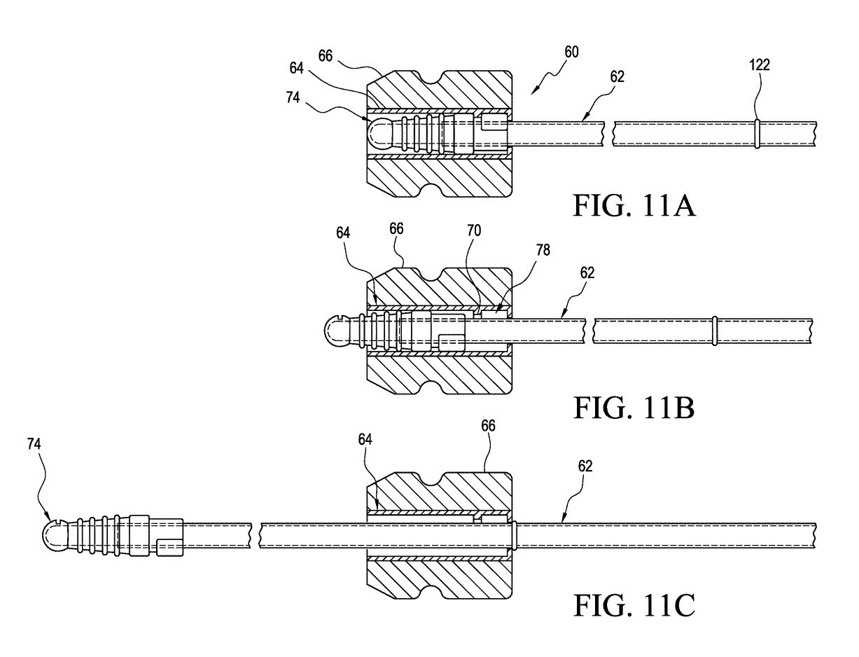

When the tip element 74 is locked into position in the hard insert 64 of foam element 66, the catheter tube 62 can be pushed and/or pulled through the vaginal canal providing locking into the cervix. This is illustrated in FIG. 11A. In this locked position, generally the catheter assembly 60 arrives at the customer. The catheter tube 62 has been rotated clockwise and is ready to be inserted into the animal. The catheter tube 62 is inserted into the cervix while in this locked position. The cervix clamps the foam in place.

To provide this initial locked position the tip element 74 is pulled towards the proximal end of the foam element 66 and the nub 80 pass through the locking rib openings 88 and are turned clockwise. The proximal nub portion 78 and the distal nub portion 76 lock inside the hard insert 64 allowing the catheter assembly 60 to be loaded into the animal and locked into the cervix like a traditional catheter. The catheter tube 62 preferably includes a stop mechanism 122 which abuts the hard insert 64 in use.

As shown in the initial unlocked position of FIG. 11B, while the cervix is holding the foam element 66 in place, the catheter tube is rotated counter clockwise by the user allowing forward movement of the catheter tube and its connected tip element relative to the foam element. The counterclockwise rotation (in the example shown, 1/4 a turn), aligns the proximal nub portion 78 allowing it to move forward of the locking rib 70. Thus, when the catheter tube 62 is rotated the catheter tube 62 can move relative to the foam element allowing it to traverse the cervix. In other words, once locked in place, rotating the catheter tube counterclockwise repositions the proximal nub portion 78 and the distal nub portion 76 to exit the hard insert 64 and be pushed into and through the cervix to service the animal.

In the final unlocked position of FIG. 11C, the catheter tube and tip element are shown completely forward of the foam element with a stop preventing further forward movement.

As in the previous embodiments, a tip element outlet end remains closed when loading the catheter tube 62 through the vaginal canal and when traversing the cervix and then automatically opens when sufficient fluid pressure in the tip element is applied, thus enhancing injection of fluid to the utero-tubal junction.

Other embodiments and configurations may be devised without departing from the spirit of the invention and the scope of the appended claims.

For example, certain features shown with respect to FIG. 1 may be combined with features described with respect to FIG. 6, etc.; and/or the features can be used independently of one another.

One such embodiment may utilize the cannula without the guide catheter, or any foam tip or assembly like FIG. 7. Since the cannula used in most embodiments is somewhat rigid, it has the ability to enter the reproductive tract and traverse the cervix without a guide. In this case, a suitable sliding mechanism to show the depth of insertion would be added to the cannula.

In other embodiments, hard insert 64 and foam element 66 can be molded entirely from one piece of material such as structural foam, or injection blow-molded in a fashion that creates a relatively hollow and soft outer shell, yet maintaining a rigid interior configuration, providing the necessary locking rib mechanisms.

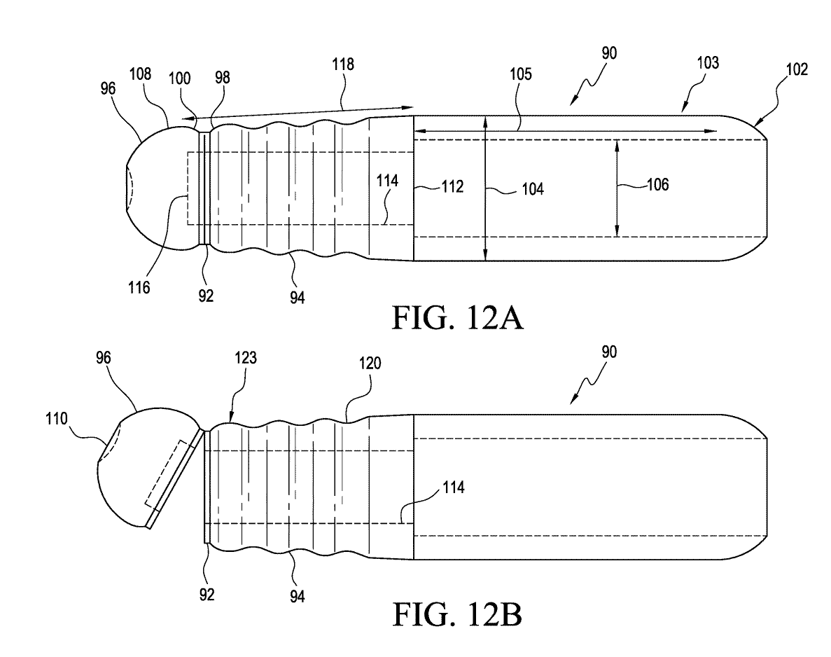

Referring now to FIGS. 12A and 12B, another embodiment of a tip element 90 illustrated which is tapered down toward the distal end. In this embodiment, the tip element 90 includes a recessed portion 92 between a main ribbed portion 94 and a bulb shaped distal end 96. As can be seen in FIG. 12B, as the distal end 96 opens to deposit fluid, its rounded edges are capable of moving away from, i.e. deflecting, off the cervical bumps and other obstructions helping to create a safe pathway into the uterus of the animal. In some embodiments, the recessed portion 92 may have an axially extending length of about 0.5 mm. The recessed portion or slicing area 92 in this embodiment is provided so that radius edges 98, 100 provide a rounded smooth surface that will remain safe if the flap portion, i.e. bulb shaped portion 96, moves out of position when deflecting off cervical folds or blockage when traversing the cervix. (This serves a similar function as the recessed portion (bend relief) 47 shown in FIG. 4.) The radius edges 98, 100 eliminate any need to specifically position the tip during the slicing process in an automated process.

As mentioned above, in one embodiment the cannula may be used without the guide catheter, or any foam tip or assembly. As discussed above, since the cannula used in most embodiments is somewhat rigid, it has the ability to enter the reproductive tract and traverse the cervix without a guide. Again, in this case, a suitable sliding mechanism is used to show the depth of insertion of the cannula.

In a preferred embodiment, the proximal end 102 of the proximal portion 103 has a 10 mm radius, an outside diameter (OD) 104 of 6.5 mm, and an inside diameter (ID) 106 of 4.3 mm. The overall length of the cylindrical portion 105 of the proximal portion 103 is welded to the cannula (not shown) and extends 16 mm to the proximal end 112 of the main ribbed portion 94. A reduced ID 114 extends through the main ribbed portion 94 into the bulb shaped distal end 96 for a distance of 10.22 mm the portion designated by reference numeral 116. The overall length of the tip element 90 is 29 mm. The length 118 of the tapered surface of the tip element from the proximal end 112 to the maximum diameter point 108 of the bulb shaped distal end 96 is 10.25 mm. The diameter of the bulb shaped distal end 96 at point 108 is 5.5 mm. An orifice 110 is provided for injecting raw materials during the molding process. Main ribbed portion 94 is configured with valleys 120 and peaks 123. The radii of the peaks and valleys may range from about .25 mm to 1 mm which assist in creating the proper flexing properties of main ribbed portion 94. The preferred slice depth at recessed portion 92 is 60-80% of the material thickness of the recessed portion 92 providing for easy opening of flap portion 96 yet providing enough strength so the flap portion 96 does not tear off and stay inside the uterus when used.

Thus, the embodiments of FIG. 6, FIG. 12A, and FIG. 12B illustrate a tip element that has a continual taper down from a proximal section to a distal (or end) section wherein a maximum diameter of the proximal section is less than the outer diameter of the guide catheter.

Although the present invention has been discussed in terms of its application with artificial insemination, it is understood that the inventive principles discussed above may be similarly utilized for embryo transfers in an animal. In such an application, the post-cervical insemination (PCAI) catheter assembly is embodied as a post-cervical embryo transfer device catheter assembly in the embodiments discussed above, using the same elements thereof. In accordance with the principles of the present invention, the large diameter opening in the distal end of tip element in all embodiments, provides for front loading of embryos into the tip, provides a clear exit path where embryos do not get stuck in very small and sideways exit holes, and provides greater depositing accuracy due to the increased flow characteristics of the present invention as explained. Embryo's cannot be front loaded into cannulas with side, or extremely small exit holes typically found with barbed inserts primarily used for decades with artificial insemination devices.

After the animal has been serviced with any embodiment of the catheter assembly with a cannula and guide catheter, the technician can leave the catheter assembly locked into the cervix and can pull back on the cannula to the distal end of the guide catheter, effectively sealing the flexible tip element against the cannula. If contractions from the animal's cervix or uterine horns try to push fluid(s) back towards the vaginal canal and out the vulva, the flap portion 18 as shown in FIG. 1 or the bulb shaped distal end 96 as shown in FIGS. 12A-12B of the tip element recloses to seal fluids from reentering the cannula, thereby preventing the loss of genetic material or backflow out the cannula and/or guide catheter.

Effectively the cervix clamps to the outer foam portion 26 sealing to the outside surface, the proximal end of the tip element forms a seal to the distal end of the cannula 12, and the flap portion of the tip element (i.e. flap portion 16 in FIG. 1/bulb shaped portion 96 in FIG. 12A) closes and returns to its original position. Traditional PCAI devices sometimes have handles 33 with caps that plug the cannula to prevent contamination from entering prior to service, and they also attempt to control backflow after servicing, but plugging the proximal end of a cannula 12 does not prevent fluids from escaping through the guide catheter 14. There is no way for traditional cannulas and tips to stop backflow directly in or at the proximal portion of the cervix, (at the point of the foam element 26) since their tips are smaller in diameter than the guide catheter and/or have open orifices on the sides or front of their tips that cannot be closed at that location preventing fluids from traveling all the way to the closed handle on the proximal end of the cannula. Some PCAI devices include a secondary stopping mechanism that slides on the cannula 12 which can lock or seal the cannula's outer surface to the ID of the guide catheter, but this allows a reservoir of space inside both the cannula and guide catheter to fill up with valuable genetic material which is wasted once the catheter assembly is removed. The present invention keeps all fluids and genetic material used during the service inside the reproductive tract where it belongs and nothing is wasted when the catheter assembly is finally removed. This effort is normally not required due to the injection and flow characteristics of the present invention which deposits the majority of fluids directly to, or closer to the utero tubal junction (UTJ), but it is commonly used in the industry as a precaution against backflow with existing products.

1. A post-cervical insemination catheter assembly, comprising:
a) a cannula;
b) a guide catheter slideably positioned about said cannula; and,
c) a tip element securely connected to a distal end of said cannula configured to receive a fluid from the cannula and dispense the fluid from a tip element outlet end, wherein said tip element outlet end remains closed when loading the cannula through a vaginal canal and when traversing a cervix of an animal and then automatically opens when sufficient fluid pressure in the tip element is applied, thus enhancing injection of the fluid to a utero-tubal junction of the animal and maintaining bio-security of fluids being transferred through the catheter assembly.

2. The catheter assembly of claim 1 wherein said tip element is about an outer surface of said cannula.

3. The catheter assembly of claim 1 wherein said catheter assembly comprises a post-cervical artificial insemination (PCAI) catheter assembly.

4. The catheter assembly of claim 1 wherein said catheter assembly comprises a post-cervical embryo transfer (PCET) catheter assembly.

5. The catheter assembly of claim 1 wherein said tip element comprises a proximal section, a distal section, and an intermediate section between said proximal section and said distal section, said proximal section being substantially cylindrical, said intermediate section being ribbed, and said distal section being bulb-shaped.

6. The catheter assembly of claim 1 wherein said tip element comprises a proximal section, a distal section, and an intermediate section between said proximal section and said distal section, said proximal section being substantially cylindrical, said intermediate section being ribbed, and said distal section being bulb-shaped, wherein a maximum diameter of said proximal section, said intermediate section and said distal section are substantially equal to an outer diameter of the guide catheter.

7. The catheter assembly of claim 1 wherein said tip element comprises a proximal section, a distal section, and an intermediate section between said proximal section and said distal section, said proximal section being substantially cylindrical, said intermediate section being ribbed, and said distal section being bulb-shaped, wherein a maximum diameter of said proximal section, said intermediate section and said distal section are greater than an outer diameter of the guide catheter to better emulate a size of a penis of the animal.

8. The catheter assembly of claim 1 wherein said tip element comprises a proximal section, a distal section, and an intermediate section between said proximal section and said distal section, said proximal section being substantially cylindrical, said intermediate section having a dual taper, and said distal section being bulb-shaped.

9. The catheter assembly of claim 1 wherein said tip element comprises a proximal section, a distal section, and an intermediate section between said proximal section and said distal section, said proximal section being substantially cylindrical, said intermediate section having a dual taper, and said distal section being bulb-shaped, wherein a maximum diameter of said proximal section and said distal section are substantially equal to an outer diameter of the guide catheter.

10. The catheter assembly of claim 1 wherein said tip element comprises a proximal section, a distal section, and an intermediate section between said proximal section and said distal section, said proximal section being substantially cylindrical, said intermediate section having a dual taper, and said distal section being bulb-shaped, wherein a maximum diameter of said proximal section and said distal section are greater than an outer diameter of the guide catheter.

11. The catheter assembly of claim 1 wherein said tip element has a continual taper down from a proximal section through a distal section.

12. The catheter assembly of claim 1 wherein said tip element has a continual taper down from a proximal section to an end section wherein a maximum diameter of said proximal section is substantially equal to an outer diameter of the guide catheter.

13. The catheter assembly of claim 1 wherein said tip element has a continual taper down from a proximal section to an end section wherein a maximum diameter of said proximal section is greater than an outer diameter of the guide catheter.

14. The catheter assembly of claim 1 wherein said tip element has a continual taper down from a proximal section to an end section wherein a maximum diameter of said proximal section is less than an outer diameter of the guide catheter.

15. The catheter assembly of claim 1 wherein said tip element outlet end comprises a slit or slits allowing it to open at said sufficient pressure.

16. The catheter assembly of claim 1 wherein said tip element outlet end comprises a breakaway membrane allowing it to open at said sufficient pressure.

17. The catheter assembly of claim 1 wherein said tip element is formed of a semi-flexible material.

18. The catheter assembly of claim 1 wherein said tip element is formed of polyvinyl chloride (PVC).

19. The catheter assembly of claim 1 wherein said tip element is formed of polyvinyl chloride (PVC) with a shore hardness of 50 on the IRHD scale.

20. The catheter assembly of claim 1 wherein said tip element is formed of polyvinyl chloride (PVC) with a shore hardness between 10 and 90 on the IRHD scale providing controllable flexural strength characteristics.

21. The catheter assembly of claim 1 wherein said tip element is formed of polyvinyl chloride (PVC) with a shore hardness between 30 and 70 on the IRHD scale providing controllable flexural strength characteristics.

22. The catheter assembly of claim 1 wherein said tip element is formed of polyvinyl chloride (PVC) and said cannula is formed of polyvinyl chloride (PVC).

23. The catheter assembly of claim 1 wherein said tip element is formed of polyvinyl chloride (PVC) and said cannula is formed of polyvinyl chloride (PVC), said tip element and said cannula being welded using high-frequency.

24. The catheter assembly of claim 1 wherein said cannula has an outside diameter of about 11/64 inches (4.368 mm).

25. The catheter assembly of claim 1 wherein said cannula includes a crimped stop, heat staked stop mechanism, handle, or injection molded stop mechanism to limit the distance it enters the animal.

26. The catheter assembly of claim 1 wherein said tip element includes a tapered wall thickness allowing the tip element to bend without collapsing onto itself following a natural pathway of the animal, thereby guiding the cannula behind it.

27. A method for providing post-cervical insemination of an animal, comprising the steps of:
a) providing a post-cervical insemination catheter assembly having:
i) a cannula;
ii) a guide catheter slideably positioned about said cannula; and,
iii) a tip element securely connected to a distal end of said cannula configured to receive fluid from the cannula and dispense the fluid from a tip element outlet end;
b) inserting the guide catheter through a vaginal canal of an animal and locking into a cervix of the animal;
c) pushing the cannula through the guide catheter to a desired position in a reproductive tract of the animal; and,
d) applying sufficient fluid pressure in the tip element to automatically open the tip element outlet end enhancing injection of fluid to a utero-tubal junction of the animal and maintaining bio-security of fluids being transferred through the catheter assembly.

28. The method of claim 27, wherein said post-cervical insemination catheter assembly comprises a post-cervical artificial insemination (PCAI) catheter assembly.

29. The method of claim 27, wherein said post-cervical insemination catheter assembly comprises a post-cervical embryo transfer (PCET) catheter assembly.

30. A post-cervical insemination catheter assembly, comprising:
a) a foam element with an inner surface
b) a hard insert with an outer surface secured to the inner surface of said foam element, said hard insert, comprising:
i) an axially extending elongated body;
ii) at least one locking rib extending from an inner surface of said axially extending elongated body;
c) a tip element, comprising:
i) a distal nub portion;
ii) a proximal nub portion including at least a first proximal nub; and at least a first associated proximal nub recess;
d) a catheter tube having a distal end securely connected to said tip element,
wherein when the tip element is locked into a locked position in the foam element the catheter tube can be pushed and/or pulled through a vaginal canal of an animal providing locking into a cervix of the animal and wherein when the catheter tube is rotated the catheter tube can move relative to the foam element allowing it to traverse the cervix of the animal, and,
wherein a tip element outlet end remains closed when loading the catheter tube through the vaginal canal and when traversing the cervix and then automatically opens when sufficient fluid pressure in the tip element is applied, thus enhancing injection of fluid to a utero-tubal junction of the animal.

31. A post-cervical insemination catheter assembly, comprising:
a) a cannula;
b) a handle connected to the cannula, said handle configured to provide attachment of a container to the cannula;
c) a tip element comprising:
i) a proximal portion securely attached to the cannula;
ii) a main ribbed portion extending from said proximal portion;
iii) a bulb shaped distal end;
iv) a recessed portion between said main ribbed portion and said bulb shaped distal end, said recessed portion providing a slicing area;
wherein said cannula is configured to receive a fluid from a container and said fluid is transferred through said proximal portion, through said main ribbed portion and dispensed through an opening created in a reduced diameter of the inner diameter of the tip element in a vicinity of said recessed portion when the bulb shaped distal end opens;
wherein said bulb shaped distal end remains seated to the main ribbed portion when loading the cannula through a vaginal canal and when traversing a cervix of an animal and then automatically opens when sufficient fluid pressure in the tip element is applied, thus enhancing injection of the fluid to a utero-tubal junction of the animal and maintaining bio-security of fluids being transferred through the catheter assembly.

32. The catheter assembly of claim 31 wherein said proximal portion is welded to the cannula.

33. The catheter assembly of claim 31 wherein said bulb shaped distal end and said main ribbed portion include rounded edges in respective portions thereof adjacent to the recessed portion.
